(19) United States
(12) Patent Application Publication
(10) Pub. No.: US 2026/0308654 A1
(43) Pub. Date: Oct. 8, 2026

(54) Method for the Production of Pharmaceutically Active Recombinant Human Low Molecular Weight Urokinase

(71) Applicant: CERBIOS-PHARMA SA, Barbengo/Lugano (CH)

(72) Inventors: Vitor Lino SOUSA, Barbengo/Lugano (CH); Luca RIGHETTI, Barbengo/Lugano (CH); Tiphanie DURFORT, Barbengo/Lugano (CH); Marco AGOSTINETTO, Barbengo/Lugano (CH); Daniele MARTELLATO, Barbengo/Lugano (CH)

(21) Appl. No.: 19/481,492

(22) PCT Filed: Mar. 29, 2024
(86) PCT No.: PCT/EP2024/058745

§ 371 Date: Nov. 4, 2025

(30) Foreign Application Priority Data
May 4, 2023 (IT) 102023000008805

(51) Int. Cl. C12P 21/00; C12N 5/071; C12N 9/72

(52) U.S. Cl. CPC C12P 21/005; C12N 5/0682; C12N 9/6462; C12N 2500/02; C12N 2500/34; C12N 2527/00; C12Y 304/21073

(57) ABSTRACT

A CHO cell line and a process for the production of recombinant human urokinase in its low molecular weight form (rh-LMW-uPA) via the cell line are described. The process has high productivity and allows the production of low molecular weight urokinase with improved characteristics in terms of safety, quality and stability.

The present invention is directed to a method for producing recombinant human urokinase in its low molecular weight form (rh-LMW-uPA), in particular a stable and active glycosylated recombinant human low molecular weight urokinase, obtained through a eukaryotic cell line using recombinant DNA techniques. The process allows to produce glycosylated recombinant human low molecular weight urokinase in the culture medium in its physiologically active (mature) form.

The technical field of the invention is the production of glycosylated recombinant human low molecular weight urokinase through genetic engineering techniques applied on eukaryotic cells of the CHO line.

Fibrinolysis is the process of dissolving blood clots thus preventing blockage of blood vessels. The fibrinolytic system is mainly regulated by proteases and protease inhibitors. The key enzyme of this system is plasmin mainly derived from inactive plasminogen via its direct activators—tissue-type plasminogen activator (tPA) and urokinase-type plasminogen activator (uPA) [Wun, T.-C., Schleuning, W.-D. & Reich, E. Isolation and Characterization of Urokinase from Human Plasma. The Journal of Biological Chemistry 257, 3276-3283 (1982)]—counteracted by specific inhibitors. The conversion of plasminogen into active plasmin via uPA determines the fibrin clot lysis, thus giving rise to fibrin degradation products [Lin, H. et al. Therapeutics targeting the fibrinolytic system. Experimental & Molecular Medicine 2020 52:3 52, 367-379 (2020)].

Human uPA, a key serine protease that has long been used clinically as a thrombolytic agent [Gurewich, V. Fibrinolytic Mechanisms of tPA, prouPA, Mutant prouPA and Their Implications for Therapeutic Thrombolysis. Cardiovascular Engineering and Technology 2013 4:4 4, 328-338 (2013); Tomasi, S., Sarmientos, P., Giorda, G., Gurewich, V. & Vercelli, A. Mutant Prourokinase with Adjunctive C1-Inhibitor Is an Effective and Safer Alternative to tPA in Rat Stroke. PLOS One 6, (2011)], is secreted into the blood by different tissues in the form of a single-chain glycosylated zymogen called pro-uPA (sc-uPA, 411 amino acids, MW 54 kDA) [Gurewich, V. Pro-urokinase: physiochemical properties and promotion of its fibrinolytic activity by urokinase and by tissue plasminogen activator with which it has a complementary mechanism of action. Semin Thromb Hemost 14, 110-115 (1988)] with very low amidolytic activity [Hedstrom, L. Serine protease mechanism and specificity. Chem Rev 102, 4501-4523 (2002)] and is activated by cleavage of the peptide bond between K¹⁵⁸-I¹⁵⁹ by plasmin creating α chains (residues 1-158) and β chains (residues 159-411) [Spraggon, G. et al. The crystal structure of the catalytic domain of human urokinase-type plasminogen activator. Structure 3, 681-691 (1995)]. The resulting fully active two-chain protein uPA (tc-uPA), also known as high molecular weight urokinase (HMW-uPA), [Primary structure of single-chain pro-urokinase—PubMed. https://pubmed.ncbi.nlm.nih.gov/2931434/; Magill, C., Katz, B. A. & MacKman, R. L. Emerging therapeutic targets in oncology: urokinase-type plasminogen activator system. http://dx.doi.org/10.1517/14728222.3.1.109 3, 109-133 (2005); Spraggon, G. et al. The crystal structure of the catalytic domain of human urokinase-type plasminogen activator. Structure 3, 681-691 (1995)] converts plasminogen to plasmin, promoting the proteolysis of fibrinogen to fibrin. Conversion to the two-chain form increases inter-domain flexibility [Behrens, M. A. et al. Activation of the Zymogen to Urokinase-Type Plasminogen Activator Is Associated with Increased Interdomain Flexibility. J Mol Biol 411, 417-429 (2011)] which allows it to interact more effectively with its substrate and improves the enzymatic activity of urokinase by approximately 100 times [Fleuryso, V., Lijnenl, H. R. & Anglb-Canosji, E Mechanism of the Enhanced Intrinsic Activity of Single-chain Urokinase-type Plasminogen Activator during Ongoing Fibrinolysis*. Journal of Biological Chemistry 268, 18554-18559 (1993)]. A further cleavage at Lys¹³⁵-Lys¹³⁶ generates the amino-terminal fragment (ATF, amino acids 1-135), which includes an EGF-like domain, i.e. the growth factor domain (GFD) (residue 1-49 of the human sequence of uPA) and the kringle domain (amino acids 50-131) (FIG. 1). The remaining 136-158 fragment, called connecting peptide (CP), is connected via a disulfide bridge to the carboxy-terminal catalytic region in the low molecular weight form (LMW-UK, amino acids 136-411, MW 33 kDa) [Gurewich, V Pro-urokinase: physiochemical properties and promotion of its fibrinolytic activity by urokinase and by tissue plasminogen activator with which it has a complementary mechanism of action. Semin Thromb Hemost 14, 110-115 (1988); Stepanova, V. v. & Tkachuk, V. A. Urokinase as a Multidomain Protein and Polyfunctional Cell Regulator. Biochemistry (Moscow) 2002 67:1 67, 109-118 (2002)], which, as it is known, maintains catalytic activity and was used as a therapeutic agent [Gates, J. & Hartnell, G. G. When urokinase was gone: commentary on another year of thrombolysis without urokinase. J Vasc Interv Radiol 15, 1-5 (2004); Gurewich, V. Pro-urokinase: physiochemical properties and promotion of its fibrinolytic activity by urokinase and by tissue plasminogen activator with which it has a complementary mechanism of action. Semin Thromb Hemost 14, 110-115 (1988)].

HMW-uPA and LMW-uPA show similar catalytic activity towards plasminogen [Sato, S. et al. High-affinity urokinase-derived cyclic peptides inhibiting urokinase/urokinase receptor-interaction: effects on tumor growth and spread. FEBS Lett 528, 212-216 (2002)].

In urokinase isolated from urine, a higher fraction of the high molecular weight form is present while urokinase obtained from cultured kidney cells contains a larger portion of the low molecular weight form [Aditiviya & Khasa, Y. P. The evolution of recombinant thrombolytics: Current status and future directions. https://doi.org/10.1080/21655979.2016.1229718 8, 331-358 (2016)].

In vivo, after its secretion, pro-uPA binds to uPAR (urokinase-type plasminogen activator receptor, uPAR, or CD87) and is converted to an active two-chain state (i.e., tc-uPA or uPA) through cleavage of the Lys¹⁵⁸-Ile¹⁵⁹ bond by neighboring membrane-bound plasmin or other proteases such as kallikrein. Receptor-bound pro-uPA is activated by plasmin faster than when free in plasma [Irigoyen, J. P., Muñoz-Cánoves, P., Montero, L., Koziczak, M. & Nagamine, Y. The plasminogen activator system: biology and regulation. Cellular and Molecular Life Sciences CMLS 1999 56:1 56, 104-132 (1999)]. Active uPA then converts plasminogen bound to the neighboring membrane into plasmin.

The amino-terminal fragment of uPA (ATF, residues 1-135) contains all the binding sites required to interact with the receptor [Barinka, C. et al. Structural Basis of Interaction between Urokinase-type Plasminogen Activator and its Receptor. J Mol Biol 363, 482-495 (2006)], a glycosylphosphatidyl-inositol (GPI)-anchored membrane protein. The obtained complex was shown to be involved in two separate biological cascades: (a) plasminogen activation, which gives rise to proteolytic activity and (b) signal transduction, which determines cell adhesion and mitogenesis. Indeed, uPAR has multiple functional roles associated with tumor progression, including tumor proliferations, apoptosis, metastasis, angiogenesis, multidrug resistance (MDR) and prognosis. High levels of uPAR expression have been detected in a variety of cancer cells, but very low levels are present in normal cells, indicating that the level of uPAR in tumor tissue is closely related to the malignancy of the tumor and the prognosis of the oncology patient [Zhai, B. T. et al. Urokinase-type plasminogen activator receptor (uPAR) as a therapeutic target in cancer. Journal of Translational Medicine vol. 20 Preprint at https://doi.org/10.1186/s12967-022-03329-3 (2022)].

rh-LMW-uPA does not contain an ATF fragment and consequently is unable to bind to the urokinase receptor (uPAR), avoiding the activation of the signaling cascade through uPAR-uPA binding.

The present invention arises from the need to replace urokinase extracted from urine and used for cleaning catheters by exploiting the plasminogen activator of the two-chain recombinant human glycosylated low molecular weight urokinase (rh-LMW-uPA), representing one of the most widely used thrombolytic agents for central venous catheter occlusions in the UK [Kumwenda, M. J., Dougherty, L., Jackson, A. & Hill, S. Prospective Audit to Study urokinase use to restore Patency in Occluded central venous catheters in hematology and oncology patients (PASSPORT 2). https://doi.org/10.1177/1129729820950997 22, 568-574 (2020)].

For this purpose, a CHO cell line was developed and deposited at the Culture Collection of Switzerland AG (CCOS) and is identified with the number CCOS 2068. The structure of human rh-LMW-uPA described here consists of two polypeptide chains, namely the α chain and β chain, connected by an interchain disulfide bond between Cys¹³ and Cys¹²¹ which is surprisingly active due to the proteolysis of the Lys²³-Ile¹ peptide bond in the culture medium of the CHO cell line identified above. The β chain contains five intrachain disulfide bonds, Cys³¹-Cys⁴⁷, Cys³⁹-Cys¹¹⁰ Cys¹³⁵-Cys²⁰⁴, Cys¹⁶⁷-Cys¹⁸³, and Cys¹⁹⁴-Cys²²². Furthermore, the β chain contains the fully active catalytic domain and in addition an N-glycosylation site within the consensus sequence Asn¹⁴⁴-Ser-Tyr as well as an O-glycosylation site within the a chain.

A substantial fraction of currently approved protein drug products requires appropriate glycosylation to exhibit optimal therapeutic efficacy. This is because glycosylation can influence a variety of physiological processes at cellular and protein levels. uPA is O- and N-glycosylated. A Fuc residue is linked via O-glycosylation to Thr-18 in the epidermal growth factor-like domain of uPA derived from cultured, urinary, and recombinant human kidney cells [Kentzer, E. J., Buko, A., Menon, G. & Sarin, V. K. Carbohydrate composition and presence of a fucose-protein linkage in recombinant human pro-urokinase. Biochem Biophys Res Commun 171, 401-406 (1990); Buko, A. M. et al. Characterization of a posttranslational fucosylation in the growth factor domain of urinary plasminogen activator. Proc Natl Acad Sci USA 88, 3992-3996 (1991)]. The localization of fucosylated Thr-18 in this domain suggests its significance in a specific receptor/ligand binding system responsible for various biological functions [The receptor-binding sequence of urokinase. A biological function for the growth-factor module of proteases—PubMed. https://pubmed.ncbi.nlm.nih.gov/3031025/]. In addition, N-glycosylation of human uPA located at Asn-302 occurs [Bansal, V. & Roychoudhury, P. K. Production and purification of urokinase: A comprehensive review. Protein Expr Purif 45, 1-14 (2006); Lenich, C., Pannell, R., Henkin, J. & Gurewich, V. The influence of glycosylation on the catalytic and fibrinolytic properties of pro-Urokinase. Thromb Haemost 68, 539-544 (1992)] in the protease domain [Steffens, G. J., Günzler, W. A., ötting, F., Frankus, E. & Flohé, L. The complete amino acid sequence of low molecular mass urokinase from human urine. Hoppe Seylers Z Physiol Chem 363, 1043-1058 (1982); Irigoyen, J. P., Muñoz-Cánoves, P., Montero, L., Koziczak, M. & Nagamine, Y. The plasminogen activator system: biology and regulation. Cellular and Molecular Life Sciences CMLS 1999 56:1 56, 104-132 (1999)]. N-glycans also contain Man, Gal, Fuc, GlcNac and Neu5Ac as well as GalNAc residues [Steffens, G. J., Günzler, W. A., ötting, F., Frankus, E. & Flohé, L. The complete amino acid sequence of low molecular mass urokinase from human urine. Hoppe Seylers Z Physiol Chem 363, 1043-1058 (1982); Mclellan, W. L., Vetterlein, D. & Roblin, R. The glycoprotein nature of human plasminogen activators. FEBS Lett 115, 181-184 (1980)]. It has been demonstrated that the biological behavior of recombinant but non-glycosylated sc-uPA is similar to that of glycosylated urinary recombinant protein [Li, X. K., Lijnen, H. R., Nelles, L., Hu, M. H., & Collen, D. Biochemical properties of recombinant mutants of nonglycosylated single chain urokinase-type plasminogen activator. Biochim Biophys Acta 1159, 37-43 (1992); The influence of glycosylation on the catalytic and fibrinolytic properties of pro-urokinase—PubMed. https://pubmed.ncbi.nlm.nih.gov/1455401/]. However, recombinant non-glycosylated sc-uPA has also been shown to be cleaved more efficiently by plasmin, to be more proteolytically active, and more rapidly inactivated by plasminogen inhibitors than its recombinant glycosylated counterpart [The influence of glycosylation on the catalytic and fibrinolytic properties of pro-urokinase—PubMed. https://pubmed.ncbi.nlm.nih.gov/1455401/]. Furthermore, sialic acid content in recombinant sc-uPA has been shown to negatively influence in vivo clearance [Henkin, J., Dudlak, D., Beebe, D. P. & Sennello, L. High sialic acid content slows prourokinase turnover in rabbits. Thromb Res 63, 215-225 (1991)].

In rabbits, recombinant prourokinase (pro-uPA) containing 2.5-3 sialic acid molecules per protein has a significantly shorter half-life than less sialylated pro-uPA. The rate at which the protein is metabolized is relatively insensitive to sialic acid content ranging from 0 to 1.5 sialic acid residues per molecule of pro-uPA.

The present invention is directed to a process for the preparation of an N-glycosylated human rh-LMW-uPA within the consensus sequence Asn₁₄₄ in the catalytic domain (FIG. 2), previously described in the literature and identified as Asn₃₀₂ (structure of pro-uPA) containing the following residues Man, Gal, Fuc, GlcNac and Neu5Ac. It has been suggested that glycosylated Asn₃₀₂ uPA is more prone to activation by plasmin and more resistant to inhibitors [The influence of glycosylation on the catalytic and fibrinolytic properties of pro-urokinase—PubMed. https://pubmed.ncbi.nlm.nih.gov/1455401/]. Furthermore, 2.2 sialic acid residues were identified per molecule of rh-LMWUK and surprisingly O-glycans derived from Core 1 AS, Core 1 MS and Core 1 DS were found on the α-chain of the connecting peptide (FIG. 2).

Currently, the commercial sale of recombinant uPA has not yet been approved by the FDA or EMA. However, various attempts to produce uPA based on recombinant DNA technologies [Aditiviya & Khasa, Y. P. The evolution of recombinant thrombolytics: Current status and future directions. https://doi.org/10.1080/21655979.2016.1229718 8, 331-358 (2016); Mican, J., Toul, M., Bednar, D. & Damborsky, J. Structural Biology and Protein Engineering of Thrombolytics. Comput Struct Biotechnol J 17, 917 (2019)] confirm on the one hand the clinical relevance of this molecule and on the other hand the need for such an approach for safety and purity reasons.

Unlike the commonly commercialized urine-extracted form of HMW urokinase, the active recombinant LMW form prepared according to the process of the present invention overcomes the potential risks of transmitting infectious agents and contaminants [The Case of Abbokinase and the FDA: The Events Leading to the Suspension of Abbokinase Supplies in the United States—Journal of Vascular and Interventional Radiology. https://www.jvir.org/article/S1051-0443 (07) 61798-9/fulltext; Hartnell, G. G. & Gates, J. The case of Abbokinase and the FDA: The events leading to the suspension of abbokinase supplies in the United States. Journal of Vascular and Interventional Radiology 11, 841-847 (2000)] and is non-antigenic thanks to the sequence of human origin secreted by eukaryotic cells unlike streptokinase [Mican, J., Toul, M., Bednar, D. & Damborsky, J. Structural Biology and Protein Engineering of Thrombolytics. Comput Struct Biotechnol J 17, 917 (2019); Ouriel, K. Safety and Efficacy of the Various Thrombolytic Agents. Reviews in Cardiovascular Medicine 2002, 3 (S2), 17-24 3, 17-24 (2002)]. Mammalian urine has a very low concentration of urokinase (10-15 ng/ml) [Vetterlein, D. & Calton, G. J. Purification of urokinase from complex mixtures using immobilized monoclonal antibody against urokinase light chain. Thromb Haemost 49, 24-27 (1983)] making the purification of the enzyme a tedious and expensive procedure. Furthermore, the multiple steps of the extraction processes generally provide low yields and make this enzyme expensive [Rouf, S. A., Moo-Young, M. & Chisti, Y. Tissue-type plasminogen activator: Characteristics, applications and production technology. Biotechnol Adv 14, 239-266 (1996)]. On the other hand, Bernik and Kwaan [Bernik, M. B. & Kwaan, H. C. Plasminogen activator activity in cultures from human tissues. An immunological and histochemical study. J Clin Invest 48, 1740-1753 (1969)] reported that cultured renal cells secrete 50-100 ng/ml uPA in vitro [Roychoudhury, P. K., Khaparde, S. S., Mattiasson, B. & Kumar, A. Synthesis, regulation and production of urokinase using mammalian cell culture: A comprehensive review. Biotechnol Adv 24, 514-528 (2006); Bansal, V. & Roychoudhury, P. K. Production and purification of urokinase: a comprehensive review. Protein Expr Purif 45, 1-14 (2006)]. Due to the extremely low concentrations of urokinase in human urine, eukaryotic cells cultured in vitro provide a superior alternative for the production and purification of urokinase. With the engineered CHO cell line of the present invention, the process disclosed herein can produce approximately 600,000 ng/ml of stable human recombinant glycosylated low molecular weight urokinase.

Several attempts to produce recombinant urokinase have been described in the literature:
-Generated and expressed in CHO cell line mutants of pro-uPA with the Lys¹⁵⁸ residue replaced by Gly¹⁵⁸ (rscu-PA-Gly158) or by Glu158 (rscu-PA-Glu158). The mutants had lower specific activity than wild-type rscu-PA and were not converted into the 2-chain form by plasmin [Nelles, L., Lijnen, H. R., Collens, D. & Holmes, W. E. The Journal of Biological Chemistry Characterization of Recombinant Human Single Chain Urokinase-type Plasminogen Activator

Mutants Produced by Site-specific Mutagenesis of Lysine 158”. Journal of Biological Chemistry 262, 5682-5689 (1987)].
Expression of recombinant pro-uPA and its deletion mutants in S. cerevisiae using the GAL7 promoter and the rennin prepeptide sequence of Mucor pusillus led to their intracellular accumulation in the endoplasmic reticulum. However, they were inactive in their native state and had to be solubilized and re-folded for biological activity [Hiramatsu, R., Horinouchi, S. & Beppu, T. Isolation and characterization of human pro-urokinase and its mutants accumulated within the yeast secretory pathway. Gene 99, 235-241 (1991)].
Glycosylated and non-glycosylated variants of pro-urokinase have been produced in the yeast Pichia pastoris. The non-glycosylated form was less stable due to proteolysis but had comparable catalytic activity to mammalian-derived recombinant pro-uPA. However, glycosylation of pro-urokinase by P. pastoris interfered with its fibrinolytic activity [Wang, P., Zhang, J., Sun, Z., Chen, Y. & Liu, J. N. Glycosylation of Prourokinase Produced by Pichia pastoris Impairs Enzymatic Activity but Not Secretion. Protein Expr Purif 20, 179-185 (2000)].
Subcultured human umbilical vein endothelial cells produced single chain uPA with low fibrinolytic activity, no fibrin specificity but high affinity to plasminogen [Booyse, F. M., Lin, P. H., Traylor, M. & Bruce, R. The Journal of Biological Chemistry. Purification and Properties of a Single-chain Urokinase-type Plasminogen Activator Form Produced by Subcultured Human Umbilical Vein Endothelial Cells*. Journal of Biological Chemistry 263, 15139-15145 (1988)].
Pro-urokinase (r-scuPA, pro-uPA) saruplase is the non-glycosylated form of recombinant scu-PA (411 amino acids) produced in E. coli. It was developed as a fibrinolytic agent since scu-PA can mediate specific clots lysis in the presence of fibrin, unlike tcu-PA which does not possess fibrin specificity and consequently increases the risk of hemorrhage after its application [Roychoudhury, P. K., Khaparde, S. S., Mattiasson, B. & Kumar, A. Synthesis, regulation and production of urokinase using mammalian cell culture: A comprehensive review. Biotechnol Adv 24, 514-528 (2006)]. Furthermore, it can be cleaved in vivo into the form of two-chain urokinase resulting in the generation of plasmin [Moser, M. & Bode, C. Pharmacology and clinical trial results of saruplase (scuPA) in acute myocardial infarction. Expert Opin Investig Drugs 8, 329-335 (1999)].
Currently, urokinase (Abbokinase®, Kinlytic™) is commercially produced using human neonatal renal cells. It contains the low molecular weight form of uPA as the active ingredient. The drug was first approved by the FDA in 1978. However, due to viral contamination concerns [The Case of Abbokinase and the FDA: The Events Leading to the Suspension of Abbokinase Supplies in the United States-Journal of Vascular and Interventional Radiology. https://www.jvir.org/article/S1051-0443 (07) 61798-9/fulltext; Hartnell, G. G. & Gates, J. The case of Abbokinase and the FDA: The events leading to the suspension of abbokinase supplies in the United States. Journal of Vascular and Interventional Radiology 11, 841-847 (2000)], the FDA has suspended the drug since December 1998 due to deviations from good manufacturing practices (GMP). Since 2002 it has been reapproved for use in cases of pulmonary embolism. However, these cells are only able to proliferate for a limited period of time (30-40 generations). The restoration of these primary cells requires extensive screening and testing of kidney donors.
Abbott Laboratories has developed a recombinant pro-urokinase known as Prolyse® and has been tested in the UK for the treatment of various thromboembolic disorders [Comparison of safety and efficacy of the various thrombolytic agents—PubMed. https://pubmed.ncbi.nlm.nih.gov/12556739/.50; Ouriel, K. et al. Thrombolysis or peripheral arterial surgery: phase I results. TOPAS Investigators. J Vasc Surg 23, 64-75 (1996)]. ruPA is completely glycosylated because it derives from a genetically engineered murine hybridoma cell line and is purified from the culture medium through a series of chromatographic steps carried out in aqueous solution. The freeze-dried therapeutic product was reconstituted with sterile water for injections. More than 90% of ruPA was in the high molecular weight form, with a specific activity of approximately 170,000 IU/mg measured by the clot lysis assay [Ouriel, K., Veith, F. J. & Sasahara, A. A. Thrombolysis or peripheral arterial Phase I results surgery]. The half-life of ruPA was found to be 7 minutes in pharmacokinetic studies performed in monkeys and is shorter than its low molecular weight counterpart. However, despite these differences, the clinical effects of the two agents are similar. The drug, however, has not been approved by the FDA yet [Roychoudhury, P. K., Khaparde, S. S., Mattiasson, B. & Kumar, A. Synthesis, regulation and production of urokinase using mammalian cell culture: A comprehensive review. Biotechnol Adv 24, 514-528 (2006)].

The Applicant had also previously developed a process for the preparation of recombinant urokinase in the culture medium of genetically modified eukaryotic cells. This process, described in EP 1 245 681, however required the use of alkanoic acids as activators and gave rise to a mixture of low molecular weight and high molecular weight uPA.

The Applicant has now found and developed an improved process for the production of recombinant human low molecular weight urokinase (rh-LMW-uPA) in a eukaryotic cell line medium (CCOS 2068) which does not require the use of alkanoic acids and which does not require the separation of the mixture of LMW-uPA and HMW-uPA since it obtains only LMW-uPA as a product.

### DESCRIPTION OF THE FIGURES

FIG. 1—Structure of pro-uPA and uPA. Pro-uPA containing a growth factor domain (GFD), a kringle domain (KD) and a catalytic serine protease domain is secreted as a single-chain precursor and undergoes catalytic cleavage between the Lys 158 and Ile¹⁵⁹ peptide bond to generate the two-chain form of uPA. By the action of a second proteolytic cleavage, the two-chain form of uPA can be further cleaved between Lys¹³⁵ and Lys¹³⁶ resulting in the formation of an inactive amino-terminal fragment (ATF) and a catalytically active low molecular weight form of uPA (LMW-uPA) [Mahmood, N., Mihalcioiu, C. & Rabbani, S. A. Multifaceted role of the urokinase-type plasminogen activator (uPA) and its receptor (uPAR): Diagnostic, prognostic, and therapeutic applications. Frontiers in Oncology vol. 8 Preprint at https://doi.org/10.3389/fonc.2018.00024 (2018)].

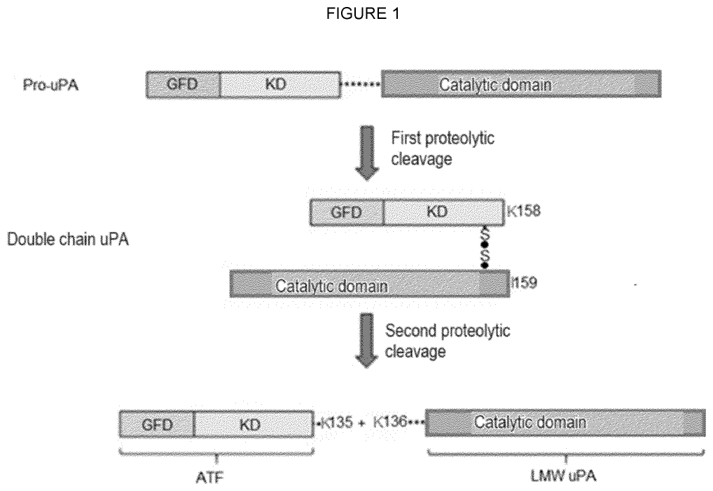

FIG. 2—Structure of the low molecular weight recombinant human urokinase obtained with the process of the present invention.

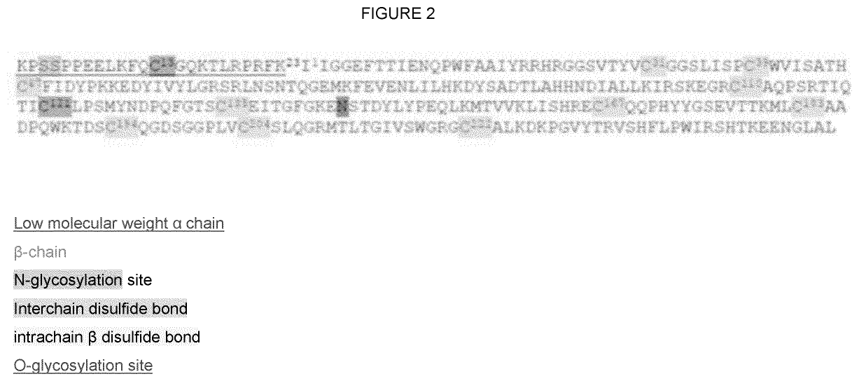

The object of the present invention is therefore a process for the production of rh-LMW-uPA in a CHO cell line (CCOS 2068) culture medium.

The general features of the process which is object of the present invention are now illustrated.

### 1. Upstream (Production Phase of the Recombinant Protein Via CHO)

The Master cell bank (MCB) and the Working cell bank (WCB) are stored in nitrogen in the vapor phase. Each cryovial contains 1 mL of concentrated cell culture, preferably 10×10⁶ cells/mL with a cryoprotective agent, preferably DMSO, at a concentration between 5-10% v/v, preferably 7.5% v/v.

The cells are thawed in a pre-warmed chemically defined culture medium, preferably CD OptiCHO (Gibco, item no. 12681011) in a 125 mL flask (Spinner Flask Corning, item no. 3152). The culture should be inoculated at 0.2-0.4×10⁶ cells/mL, preferably 0.3×10⁶ cells/mL and incubated at 37° C., 8.0% CO₂, saturated rH (preferably >85%) at 40 rpm of stirring.

The cell culture is checked periodically, measuring VCD and viability by cell counting, using trypan blue exclusion staining.

A subculture is created when the culture density is at least 1.0×10⁶ cells/mL, preferably between 1.0×106 and 2.0×10⁶ cells/mL. The subculturing procedure consists of diluting the culture in new chemically defined medium, preferably CD OptiCHO, to re-inoculate at 0.2-0.4×10⁶ cells/mL, preferably 0.3×10⁶ cells/mL.

Cells are expanded in a 1 L flask (Spinner Flask Corning item no. 3561) by subculturing as described above.

The bioreactor is inoculated at 0.1-0.4×10⁶ cells/mL, preferably 0.3×10⁶ cells/mL. The inoculum volume can be 1:3-1:8 compared to the final inoculated volume. The bioreactor culture settings are:
pH between 7.0-7.2, preferably 7.05
Dissolved oxygen between 40 and 60%, preferably 50%
Temperature between 35 and 37.5° C., preferably 37.0° C.
Stirring 0.15-0.45 m/s (angular velocity), preferably 0.35 m/s
Gas flow 0.02-0.2 vvm, preferably 0.075 vvm.

The cells are cultured for 2 to 4 days, preferably 3 days and after this culture period the grown cells can be used as an inoculum for a larger bioreactor or for production purposes by changing the culture settings to:
pH between 6.90-7.05, preferably 6.90
dissolved oxygen between 40 and 50%, preferably 40%
Temperature between 35 and 37.5° C., preferably 37.0° C.
Stirring 0.15-0.45 m/s (peak speed), preferably 0.35 m/s
Gas flow 0.02-0.2 vvm, preferably 0.075 vvm

Continuous addition of Ex-Cell Advanced Feed 1G (SAFC item #24368C-10L) starts at a flow rate of 2-8% of the initial volume per day, preferably 4%.

If the culture is used as an expansion, one proceeds as described above for the production phase.

The culture is continued maintaining the glucose concentration >1.0 g/L, preferably 2-11 g/L.

It is harvested on day 12-15 after inoculation, preferably on day 14.

At the end of the culture, the cell supernatant is recovered by centrifugation or depth filtration. The latter approach is carried out using Millistak+Pro HC or Millistak+HC depth filters (preferably Millistak+Pro HC). The cell culture should be filtered at 100 LMH and the culture load is 90-680 L/m² with a sizing of 2-10 g/m² (wet biomass). The bacterial load is then reduced by filtration.

### 2. Downstream (Purification Phase of the Recombinant Protein)

The pH of the supernatant is adjusted to pH 5.4-8.0, preferably 5.5 by slow addition of 5% v/v acetic acid, 100 mM NaCl. The pH correction should preferably be carried out in 20 minutes.

After pH correction, the supernatant is filtered through a double layer filter (0.5-0.2 μm), preferably Merck Express SHC 0.45-0.2 μm with a flow rate range of 250-1700 LMH.

The supernatant is loaded onto a resin conjugated with the pABA (p-amino benzamidine) ligand using a linear loading flow rate range of 90-150 cm/h, preferably 100 cm/h. The resin should be pre-equilibrated with an appropriate buffer that has a pH between 5.5 and 8.0, preferably 100 mM Na-Acetate, 10 mM CaCl₂), PH 5.5 buffer. Due to the high binding specificity between the p-ABA ligand and the protein, based on the active site of the serine proteases, this chromatographic phase is considered the first phase of viral reduction.

After the loading step, resin washing is required which should be carried out using a linear loading flow rate range of 90-150 cm/h, preferably 100 cm/h for 2.5 CV employing an appropriate pad with a pH between 5.5 and 8.0, preferably 100 mM Na-acetate buffer, 10 mM CaCl₂), PH 5.5.

Subsequently, a washing step should be performed using a linear load flow rate range of 90-150 cm/h, preferably 100 cm/h for 2.5 CV with an appropriate high ionic strength buffer with a pH between 5.5 and 8.0, preferably 100 mM Na-acetate buffer, 10 mM CaCl₂), 900 mM NaCl, pH 5.5.

After the high ionic strength washing step, a re-equilibration is performed using a linear load flow rate range of 90-150 cm/h, preferably 100 cm/h for 2.5 CV with an appropriate buffer with a pH between 5.5 and 8.0, preferably 100 mM Na-Acetate buffer, 10 mM CaCl₂), pH 5.5.

The purified intermediate sample is eluted at acidic pH between 2.7 and 4.0 with a Glycine-HCl or Acetate buffer, preferably 100 mM Glycine-HCl buffer pH 2.7. Sodium chloride may be added to these buffers up to 400 mM. The expected yield of the phase is >60% and is evaluated on the enzymatic activity.

Column efficiency can be restored with 1M acetic acid, 20% v/v ethanol and the column can be stored in 76 mM NaCl, 24% v/v ethanol.

The intermediate eluted from the pABA column is further adjusted to pH 3.4-4.0, preferably 4.0. The pH correction is carried out by adding strong acids or bases, preferably 500 mM NaOH or 5% v/v HCl over 20 minutes. Acid elution combined with storage time is used as a second reduction of viral contamination.

Viruses inactivation at low pH can be carried out between 4° C. and 25° C. for 2-20 hours, preferably at 4° C.

The intermediate sample is further purified on a strong cation exchange resin, preferably Fractogel EMD SO3- (Merck, article no. 1.16882), after equilibration with an appropriate buffer at pH 5.0-7.4, preferably 20 mM Na-Phosphate buffer pH 6.0, at a linear flow rate of 100-200 cm/h, preferably 200 cm/h.

Washing steps are required and should be performed using a linear flow rate range of 100-200 cm/h, preferably 200 cm/h for 2.5 CV with an appropriate buffer with a pH between 5.0 and 7.4, preferably 20 mM Na-Phosphate buffer, pH 7.4.

The intermediate product is eluted using a linear flow rate range of 100-200 cm/h, preferably 200 cm/h for 2.5 CV with an appropriate high ionic strength buffer with a pH between 5.0 and 7.4, preferably 20 mM Na-Phosphate buffer, 350 mM NaCl, pH 7.4. The yield of the phase is >80% evaluated on the enzymatic activity.

The efficiency of the column is restored with 20 mM Na-Phosphate, 1 M NaCl, pH 7.4. In-place cleaning of the column can be carried out with 500 mM NaOH and preservation is achieved with 150 mM NaCl, 20% v/v ethanol solution.

The intermediate eluted by cation exchange is filtered to remove viruses, intended as a third step in reducing viral contamination. Viresolve shield (Merck item no. VPPS101NB1) and Viresolve pro (Merck item no. VPMD101NB1) were used for filtration at a constant pressure of 1.8-2.2 bar, preferably 2.0 bar. Alternatively, a Sartopore 0.1 μm (Sartorius, item no. 5443538M8M7FFA) followed by a Planova 20N (Asahi Kasei, item no. 20NZ-300) can be used for constant pressure filtration of 1.8-2.2 bar, preferably 2.0 bar.

### 3. Formulation

The intermediate after filtration for viral removal is further formulated by tangential flow filtration (TFF) using a feed flow rate range of 200-400 LMH, preferably 360, maintaining a transmembrane pressure of 0.8-1.2 bar, preferably 1.0. A regenerated cellulose membrane can be used. The cut off should be less than 10 kDa, preferably 5 kDa.

7 volumes are commonly needed to complete the formulation. The buffer should be Na-Phosphate or Na-Acetate at pH 4.0-7.0, with the possible addition of EDTA up to 2.5 mM and Mannitol up to 6% (w/v).

The achievable range of final concentration of the pharmaceutical substance is between 0.5 and 10 g/L, stored between 4° and −80° C.

### 4. Specifications of the Drug Substance

The protein concentration should be between 0.5 and 10 g/L.

The specific activity of the final pharmaceutical substance is between 200,000 and 300,000 IU/mg.

The monomer purity determined by HPLC-SEC is >98.0%

The total sialic content is ≤5%, the HCPs content <100 ppm and the residual DNA<10 ppm.

When comparing the essential features of the process object of the invention with the process described in EP 1 245 681, the advantages of the process according to the present invention appear clearly evident.
The use of the new cell line allows the production of only low molecular weight urokinase with improvement of the product in terms of safety, quality and stability
Activation of the enzyme to convert sc-uPA into to-uPA does not require the addition of alkanoic acids to the culture medium. Activation can occur in the presence of proteases, for example uPA itself.
Productivity is significantly increased (7000 IU/mL vs 120000 IU/mL).
The obtained product has a high content of sialic acid which increases the stability of the product in the patient.

The invention will now be illustrated in greater detail in the following examples which, however, are not to be understood as limiting.

### EXAMPLES

List of abbreviations used in the examples:
MCB: Master cell bank
WCB: Working cell bank
DMSO: Dimethyl sulfoxide
CHO: Chinese Hamster Ovary cells
rH: Relative humidity
VCD: Viable Cell Count
DO: Dissolved Oxygen
LMH: liter per square meter per hour
CV: Column Volume
CIP: Cleaning in place
TMP: Transmembrane Pressure
HCPs: Host cell proteins

### Materials

| Reference | Producer | Catalog numbers |
|---|---|---|
| CD Opti CHO media | Gibco | 12681011,12681029, |
| | | A1122204, A1122201, |
| | | A1122205. |
| Excell advanced Feed | SAFC | 24368C-10L, 24368C-50L |
| 1G | | |

### Example 1

One vial of MCB or WCB was thawed and inoculated at 0.3×10⁶ cells/mL in CD Opti CHO and cultured at 37.0° C., 140 rpm, 85% rH, 8.0 CO₂. The culture was monitored regularly for VCD and viability. The culture was split by dilution with fresh medium when the VCD was between 1.0 and 3.0×10⁶ cells/mL. Seeding after splitting was 0.3×10⁶ cells/mL.

The cell culture volume was expanded to the bioreactor inoculum and then the Multifors MUF 2C Pack was inoculated at 0.3×10⁶ cells/mL into CD Opti CHO. CHO cells were cultured at 37.0° C., 50% DO, pH 7.05 with 0.29 m/s shaking angular velocity and 0.075 vvm for 3 days.

On the third day, culture feeding was started with Ex-Cell advanced feed 1G at a rate of 4% (of initial volume) per day.

Concomitant with the start of feeding, the culture parameters were changed to: 37.0° C., 50% DO, pH 7.05, 0.31 m/s angular shaking speed and 0.100 vvm. The culture was continued for up to 14 days and at the end the supernatant was collected.

### Example 2

One vial of MCB or WCB was thawed and seeded at 0.3×10⁶ cells/mL in CD Opti CHO and cultured at 37.0° C., 140 rpm, 85% rH, 8.0 CO₂. The culture was monitored regularly for VCD and viability. The culture was split by dilution with fresh medium when the VCD was between 1.0 and 3.0×10⁶ cells/mL. The inoculum after division was 0.3×10⁶ cells/mL.

The cell culture volume was expanded to the bioreactor inoculum and then the Multifors MUF 2C Pack was inoculated at 0.3×10⁶ cells/mL into CD Opti CHO. CHO cells were cultured at 37.0° C., 50% DO, PH 7.05 with 0.29 m/s shaking angular velocity and 0.075 vvm for 3 days.

On the third day, feeding of the culture started with Ex-Cell advanced feed 1G was added at a rate of 4% (of initial volume) per day.

After the feeding phase, the culture parameters were changed and set to: 37.0° C., 50% DO, pH 6.90, 0.31 m/s angular shaking speed and 0.075 vvm. The culture was continued for up to 14 days and at the end the supernatant was collected.

### Example 3

The supernatant was acidified to pH 6.5 and loaded onto a pABA resin to perform affinity chromatography. The column resin was equilibrated with 20 mM Na-Phosphate buffer, 400 mM NaCl pH 6.5 before loading the protein onto the column. The linear flow velocity was set to 90 cm/h; after the loading phase, washing with 20 mM Na-Phosphate buffer, 400 mM NaCl pH 6.5 was necessary. The product bound to the resin was further eluted with 100 mM glycine pH 2.7. CIP and column storage were performed according to the supplier's instructions.

### Example 4

The supernatant was basified to pH 8.0 and loaded onto a pABA resin to perform affinity chromatography. The column resin had to be equilibrated with 20 mM Na-Phosphate buffer, 400 mM NaCl pH 6.5 before protein loading. The linear flow velocity was set to 30 cm/h; after the loading phase, washing with 20 mM Na-Phosphate buffer, 100 mM NaCl pH 6.5 was necessary. The product bound to the resin was further eluted with 100 mM acetic acid, 100 mM NaCl pH 4.0. CIP and column storage were performed according to the supplier's instructions.

### Example 5

The intermediate obtained from the capture step was subjected to pH correction to 5.0 with 0.5 M sodium hydroxide solution and was subsequently loaded onto a strong cation exchange resin to perform ion exchange chromatography. The column resin was equilibrated with 20 mM Na-Phosphate buffer pH 6.0 before protein loading. The linear flow velocity was set to 100 cm/h. After the loading phase, two washings were performed with 20 mM Na-Phosphate buffer pH 6.0 and then with 20 mM Na-Phosphate buffer pH 7.4. The product was further eluted with 20 mM Na-Phosphate buffer, 350 mM NaCl pH 6.0. CIP and column storage were performed according to the manufacturer's instructions.

### Example 6

The intermediate obtained from the capture step was subjected to pH correction to 5.0 and was subsequently loaded onto a strong cation exchange resin to perform ion exchange chromatography. The column resin was equilibrated with 20 mM Na-Phosphate buffer pH 6.0 before protein loading. The linear flow velocity was set to 100 cm/h. After the loading phase, a washing was performed with 20 mM Na-Phosphate buffer pH 6.0. The product was eluted with 20 mM Na-Phosphate buffer, 350 mM NaCl pH 7.0. CIP and column storage were performed according to the manufacturer's instructions.

### Example 7

The ion exchange chromatography eluate was loaded onto a regenerated cellulose TFF membrane module with a 10 kDa cut off considering a loading of 200 g/m². The recirculation flow rate was set to 300 LMH and the TMP to 1.0 bar. Seven volumes of 50 mM Na-acetate pH 5.5 formulation buffer were used for the final formulation. Diafiltration can be performed at protein concentrations between 5 and 10 g/L. The final drug substance was concentrated to 5.0 g/L and frozen below −20° C.

### Example 8

The ion exchange chromatography eluate was loaded onto a regenerated cellulose TFF membrane module with a 5 kDa cut off considering a loading of 50 g/m². The recirculation flow rate was set to 360 LMH and the TMP to 1.0 bar. Seven volumes of 10 mM Na-phosphate, 2.5 mM EDTA, 6% v/v pH 7.0 formulation buffer were used for the final formulation. Diafiltration can be performed at protein concentrations between 5 and 10 g/L. The final drug substance was concentrated to 0.5 g/L and frozen below −20° C.

1. A CHO cell line deposited at the Culture Collection of Switzerland AG (CCOS) and identified under the number CCOS 2068.

2. A process for the production of recombinant human urokinase in its low molecular weight form (rh-LMW-uPA) in a culture medium of the cell line CCOS 2068 of claim 1 comprising the steps of cell culture, recover of the supernatant by centrifugation or depth filtration, purification on resin of the resultant urokinase and formulation by tangential flow filtration.

3. The process according to claim 2 wherein the cell culture is carried out under the following conditions:
pH between 6.90-7.05, preferably 6.90
Dissolved oxygen between 40 and 50%, preferably 40%
Temperature between 35 and 37.5° C., preferably 37.0° C.
Stirring 0.15-0.45 m/s (tip speed), preferably 0.35 m/s
Gas flow 0.02-0.2 vvm, preferably 0.075 vvm
and maintaining glucose concentration >1.0 g/L, preferably 2-11 g/L.

4. The process according to claim 2 wherein the recovery of the supernatant is carried out by filtration at 100 LMH and the culture load is 90-680 L/m² with a sizing of 2-10 g/m² (wet biomass).

5. The process according to claim 2 wherein the pH of the supernatant is adjusted at pH 5.4-8.0, preferably 5.5, by slow addition of 5% v/v of acetic acid, 100 mM NaCl, preferably in 20 minutes, before the purification on resin.

6. The process according to claim 2 wherein the supernatant is purified on a resin conjugated with the pABA (p-amino benzamidine) ligand pre-equilibrated with a buffer with a pH between 5.5 and 8.0, preferably 100 mM Na-Acetate, 10 mM CaCl₂), pH 5.5 buffer.

7. The process according to claim 2 wherein the purified urokinase is eluted from the resin at acidic pH between 2.7 and 4.0 with a Glycine-HCl or Acetate buffer, preferably 100 mM Glycine-HCl pH 2.7 and the eluted product is further adjusted to pH 3.4-4.0, preferably 4.0, by addition of alkaline solution, preferably 500 mM NaOH in 20 minutes.

8. The process according to claim 6 wherein after the purification on a resin conjugated with the pABA ligand, the urokinase is further purified on a strong cation exchange resin by washing with a low ionic strength buffer at pH 7.4, preferably 20 mM Na-Phosphate, and by eluting using a linear flow rate range of 100-200 cm/h, preferably 200 cm/h for 2.5 CV with a high ionic strength buffer with a pH between 5.0 and 7.4, preferably 20 mM Na-Phosphate, 350 mM NaCl, pH 7.4.

9. The process according to claim 2 wherein the formulation by tangential flow filtration (TFF) is carried out by using a feed flow rate range of 200-400 LMH, preferably 360, maintaining a transmembrane pressure of 0.8-1.2 bar, preferably 1.0, with a cut off lower than 10 kDa, preferably 5 kDa.

10. The process according to claim 9 wherein Na-Phosphate or Na-Acetate buffer at pH 4.0-7.0 is used, with the optional addition of EDTA up to 2.5 mM and Mannitol up to 6% (w/v).

11. The process according to claim 2 wherein the specifications of the obtained product are:
protein concentration between 0.5 and 10 g/L, stored between 4° and −80° C.
specific activity between 200,000 and 300,000 IU/mg
monomer purity determined by HPLC-SEC >98.0%


SEQUENCE LISTING (1 sequence)

SEQ ID NO: 1 - protein, 276 aa, unidentified
KPSSPPEELKFQCGQKTLRPRFKIIGGEFTTIENQPWFAAIYRRHRGGSVTYVCGGSLISPCWVISATHCFIDYPKKEDYIVYLGRSRLNSNTQGEMKFEVENLILHKDYSADTLAHHNDIALLKIRSKEGRCAQPSRTIQTICLPSMYNDPQFGTSCEITGFGKENSTDYLYPEQLKMTVVKLISHRECQQPHYYGSEVTTKMLCAADPQWKTDSCQGDSGGPLVCSLQGRMTLTGIVSWGRGCALKDKPGVYTRVSHFLPWIRSHTKEENGLAL